SEQUENCE LISTING (22 sequences)

SEQ ID NO: 1 - DNA, 38 bp, Homo sapiens
tcttttcagagccactcacgcttccagagaaagagcct

SEQ ID NO: 2 - DNA, 38 bp, Homo sapiens
tcttttcagagccactcacacttccagagaaagagcct

SEQ ID NO: 3 - DNA, 19 bp, Artificial Sequence
  Description of Artificial Sequence Probe
agaaaagtctcggtgagtg

SEQ ID NO: 4 - DNA, 24 bp, Artificial Sequence
  Description of Artificial Sequence Probe
tatatagatattagatataaagaa

SEQ ID NO: 5 - DNA, 21 bp, Artificial Sequence
  Description of Artificial Sequence Probe
aacccctaaaatatctaaaat

SEQ ID NO: 6 - DNA, 20 bp, Artificial Sequence
  Description of Artificial Sequence Probe
agctgttcgtgttctatgat

SEQ ID NO: 7 - DNA, 19 bp, Artificial Sequence
  Description of Artificial Sequence Probe
ctccacacggcgactctca

SEQ ID NO: 8 - DNA, 20 bp, Artificial Sequence
  Description of Artificial Sequence Probe
ggaagagcagagatatacgt

SEQ ID NO: 9 - DNA, 20 bp, Artificial Sequence
  Description of Artificial Sequence Probe
ggcctgggtgctccacctgg

SEQ ID NO: 10 - DNA, 21 bp, Artificial Sequence
  Description of Artificial Sequence Probe
aaggcactcttgcctacgcca

SEQ ID NO: 11 - DNA, 21 bp, Artificial Sequence
  Description of Artificial Sequence Probe
aggcactcttgcctacgccac

SEQ ID NO: 12 - DNA, 20 bp, Artificial Sequence
  Description of Artificial Sequence Probe
aacttgtggtagttggagct

SEQ ID NO: 13 - DNA, 21 bp, Artificial Sequence
  Description of Artificial Sequence Probe
actttgtggtagttggagctg

SEQ ID NO: 14 - DNA, 20 bp, Artificial Sequence
  Description of Artificial Sequence Probe
actggtggtggttggagcag

SEQ ID NO: 15 - DNA, 19 bp, Artificial Sequence
  Description of Artificial Sequence Primer
gggctggataaccttggct

SEQ ID NO: 16 - DNA, 20 bp, Artificial Sequence
  Description of Artificial Sequence Primer
gtcacataccccagatcaca

SEQ ID NO: 17 - DNA, 21 bp, Artificial Sequence
  Description of Artificial Sequence Primer
ccttggtctttccttgtttga

SEQ ID NO: 18 - DNA, 21 bp, Artificial Sequence
  Description of Artificial Sequence Primer
tctggcttgaaattctactgg

SEQ ID NO: 19 - DNA, 47 bp, Homo sapiens
  mutation (22): c to g
cagctgttcgtgttctatgatcatgagagtcgccgtgtggagccccg

SEQ ID NO: 20 - DNA, 47 bp, Homo sapiens
  mutation (26): g to c
cggggctccacacggcgactctcatgatcatagaacacgaacagctg

SEQ ID NO: 21 - DNA, 47 bp, Homo sapiens
  mutation (22): g to a
gggaagagcagagatatacgtgccaggtggagcacccaggcctggat

SEQ ID NO: 22 - DNA, 48 bp, Homo sapiens
  mutation (27): c to t
atccaggcctgggtgctccacctggtcacgtatatctctgctcttccc

(19) United States
(12) Patent Application Publication
(10) Pub. No.: US 2003/0186262 A1
(43) Pub. Date: Oct. 2, 2003

(54) Novel dna chips

(76) Inventor: Fabrice Cailloux, Clermont-Ferrand (FR)

Correspondence Address: FOLEY AND LARDNER, SUITE 500, 3000 K STREET NW, WASHINGTON, DC, 20007, US

(21) Appl. No.: 10/220,507

(22) PCT Filed: Mar. 1, 2001
(86) PCT No.: PCT/FR01/00604

(30) Foreign Application Priority Data
Mar. 1, 2000 (FR) 00/02614

(51) Int. Cl. C12Q 1/68; C12P 19/34

(52) U.S. Cl. 435/6; 435/91.2

(57) ABSTRACT

The invention concerns a DNA chip system for detecting mutation in a target nucleic acid such that only the DNA comprising the mutation remains on the chip at the end of the process. The invention concerns a method which consists in adding a complementary αS-phosphothioatedesoxynucleotide of the mutation is added by means of DNA polymorase at the 3′ end of the probe hybridised with the target nucleic acid and in adding an exonuclease so that only the elongated probes are not degraded. The detection of the presence or absence of mutation is carried out by directly or indirectly measuring the presence or the absence of DNA in a specific site on the chip. Advantageously, the chip comprises ISFET transistors or piezoelectric transducers.

The present invention relates to a DNA chip system for detecting a mutation in the target nucleic acid such that only the DNA comprising the mutation remains on the chip at the end of the process. The invention relates to a method in which an αS-phosphothioatedeoxynucleotide complementary to the mutation is added by means of a DNA polymerase to the 3′ end of the probe hybridized with the target nucleic acid and in which an exonuclease is added so that only the elongated probes are not degraded. The detection of the presence or absence of the mutation is carried out by directly or indirectly measuring the presence or absence of DNA in a specific site on the chip. Advantageously, the chip comprises ISFET type transistors or piezoelectric transducers.

Mutations in the germ cells or in somatic lines can have formidable consequences on the body by causing, for example, inherited genetic diseases or the appearance of cancer. The effect of a mutation closely depends on its localization in the DNA. In the case of a mutation in a coding region, the loss of the function of the encoded protein may be observed. If the mutation is present in a regulatory region, the expression of the DNA may be abolished or increased. A mutation in a gene involved in cancer at the level of the germ line does not necessarily mean that the individual concerned will effectively contract a tumor but merely that its risk is increased. In addition, when it is sought to diagnose the invasive potential of an already established tumor, it is not known in advance what mutations to expect because they may be present on several genes or at several sites of the same gene. Consequently, it appears necessary to be able to simultaneously detect numerous mutations.

The need for a technique for detecting mutations, for typing or alternatively for studying polymorphism is increasingly felt in industry either to allow the discovery of novel biological targets of interest, or to know precisely the genetic profile of a tumor or of a patient and envisage suitable therapy. This need has led to the development of various techniques such as LCR, SSCP and RFLP, but they do not allow a systematic search for numerous mutations in a sample.

The objective forming the basis of the present invention was to develop a technique allowing simultaneous determination of several nucleotides to be identified and consequently the diagnosis of mutations and of polymorphisms of genes, or the identification of pathogenic or genetically modified microorganisms. More specifically, the problem consists in a compilation of various biochemical, electronic or optical techniques within the same device which would be particularly easy to use, which could generate signals with a low noise/signal ratio without requiring a tedious and a complicated interpretation as a result. It is also important to provide a device which is as integrated as possible and has a low cost.

The DNA chips could satisfy the abovementioned problems, but such as they are provided in the state of the art, they have inherent limitations which is slowing down their large-scale exploitation.

A chip consists of a multitude of nucleic probes precisely attached to defined sites on a solid support provided in the form of flat or porous surfaces composed of various materials allowing such an attachment.

Up until now, the choice of support was determined by its capacity to allow the attachment of the probes. Materials such as glass, silicon or polymers are commonly used in the state of the art. The probes are grafted onto these surfaces during a first step called “functionalization” in which an intermediate layer of reactive molecules are added in order to capture or bind the probes.

Glass is a choice material since it is inert, nonpolar and mechanically stable. It has been used in a method for the in situ synthesis of oligonucleotides by photochemical targeting developed by the company Affymetrix. This technique consists in using a glass surface activated by addition of silane carrying NH₂ or OH groups; Sheldon E. L. (1993) Clin. Chem. 39(4), 718-719.

Another method consists in covering the glass surface with poly-L-lysine, placing the probes and then carrying out the graft by exposure to UV radiation. Polymers such as the polypyrroles developed by CIS Biointernational may also be mentioned.

Once the probes have been attached to the solid support, the DNA derived from samples is allowed to hybridize under predetermined conditions. The base composition of the duplex is an essential element influencing its stability which depends strictly on the melting temperature (Tm). When it is sought to detect point mutations, mismatches cause a drop in the Tm, which has the consequence of eliminating nucleic acids which are not totally hybridized during the washing step. Thus it is virtually impossible to seek to simultaneously detect several mutations in several genes of interest because the Tm values vary from one duplex to the other. Furthermore, the length of the probes represent a nonnegligible technical difficulty when it is desired to simultaneously detect numerous mutations with the aid of various probes of different length.

As regards the step for detecting the hybridizations, the use of fluorescent molecules, such as fluorescein, constitutes the most common labeling method. This method allows direct or indirect revealing of the hybridization and the use of various fluorochromes in the same experiment. However, it remains expensive, because it requires the use of fairly cumbersome devices for reading the emitted wavelengths and for interpreting the signal.

The detection of hybridization may also be carried out using radioactive markers. However, this technique does not make it possible to obtain a satisfactory definition when it is sought to miniaturize the chips.

An alternative approach consists in using the properties of semiconductor materials. For example, it is possible to choose a solid support-based on silicon (Si) coated with a dielectric (SiO₂) onto which the probes are attached. Under certain suitable polarization conditions, a current, sensitive to the semiconductor charge modifications, normally circulates from the source to the drain. The hybridization between the probes and the DNA of the sample causes a modification of the semiconductor charge density at the Si/SiO₂ interface. This variation may be measured and makes it possible to detect the specific hybridization between probes and target nucleic acids; Souteyrand et al. (1995) Lettre des Sciences Chimiques 54, 9-11. This technique is used by the IFOS laboratory of the Ecole Centrale of Lyon.

Another possibility is the use of the chip developed by Bechman Instruments (Permittivity Chips™) which incorporates the dielectric dispersion due to the negative charges of the phosphate groups present in the nucleotide backbone. This phenomenon, which depends on the length of the DNA molecule, may be quantified by the frequency of relaxation of the molecule. This parameter indeed varies by a factor of 100 when the quantity of DNA varies by a factor of 10; Beattie K. et al (1993) Clin Chem 39 (4), 719-721. In this technology, an impedance analyzer is used to measure the energy absorbed by the probes when they are paired.

The chips intended for analyzing mutations should be capable of analyzing, with the aid of probes, each base of a sequence already known or of detecting mutations identified beforehand as being involved in diseases such as cancer.

In the state of the art, these probes are described as comprising a part homologous to the wild-type sequence and a modification (substitution, deletion, addition) localized in the middle of the sequence in order to standardize the hybridization conditions. In the case of base substitution analysis, the probes are organized into tetrads, sets of tour elements in which one of the probes possesses, in a central position, the base homologous to the nucleotide present in the wild-type sequence; the other three probes containing the other three possible bases. This analysis in extenso is described in Chee M. et al. (1996) Science 274, 610-613. According to this technique, a DNA chip was developed to detect heterozygous mutations in the BRCA1 gene by measurement of the fluorescence. This system comprises about 10⁵ oligonucleotides allowing the detection of substitutions and of insertions of single bases, as well as deletions 1 to 5 nucleotides long. The system for analyzing the hybridizations is based on labeling with two colors (green by fluorescein and red by a phycoerythrin and streptavidin combination); Hacia J G et al. (1996) Nature Genet 14, 441-447.

As mentioned above, the constitution of the chips should be improved because the analysis of hybridizations is made difficult by photochemical targeting which produces impurities and by variations in the stability of the heteroduplexes. Furthermore, the devices currently available on the market are relatively expensive. Finally, this system is limited by the fact that a step of amplification of the samples is necessary if it is desired to obtain a detectable signal. A review on DNA chips is presented in Gramsey Graham “DNA Chips State of the Art” Nature Biotechnology vol. 16, January 1998, in Hinfray G. “Les puces à ADN” [DNA chips] Biofutur, April 1997 No. 166, Journal No. 91 and in Marshall A. and Hodgson J.; Nature Biotechnology Vol. 16, January 1998.

In the context of the present invention, a DNA chip system has been developed which is based on the specific hybridization of the probe (serving in the present case as oligonucleotide primer) with the target DNA, the extension the probe with selective addition of at least one oligonucleotide derivative to the 3′ end of the primer complementary to the target DNA; the primer thus extended being resistant to digestion by an exonuclease, in particular by exonuclease III. It is possible, for example, to add an αS-phosphothioate-deoxynucleotide by means of a DNA polymerase, which prevents exonuclease III from digesting the duplex.

Thus, the DNA remains present at a specific site on the chip only when the following conditions are met:

a) hybridization between the probe and the target DNA of the sample, and

b) presence of a complementary base in the target DNA allowing the incorporation of αS-phosphothioate-deoxynucleotide into the probe, which prevents its degradation by nuclease.

In the case where the probe does not hybridize with the target DNA, there is elimination of the probe at a specific site (microwells and the like) . Likewise, if the target DNA does not contain the base complementary to the given αS-phosphothioatedeoxynucleotide, the latter is not incorporated and the probe is then digested by the nuclease.

Results which are simple to interpret are thus obtained since they are only of two types:

DNA present (1)

or DNA absent (0).

This technique, associated with an electronic solid support, makes it possible to measure the difference in charge, conductance, resistance, impedance or any other effect of electrical variation, of field effect variation or alternatively any mass variation causing an electrical variation (piezoelectric transducer) on the solid support. For example, such a support may be a semiconductor system, in particular an ISFET (ion sensitive field effect transistor) system. This system therefore captures simple signals 0 (no DNA) or 1 (DNA) of the binary type which may be directly transmitted to a data processing system, in particular to a computer.

It is also possible to detect the presence of DNA at a specific site of the chip by optical reading (modification of the optical properties of the support such as refraction, variation of the density, or measurement of the fluorescence) for example by coupling the device to a CCD camera In such a system, the results are easy to interpret because they are limited to the results DNA present (1) or DNA absent (0) and all the signals between 1 and 0 obtained up until now in the state of the art are eliminated.

The consequent advantages of this system consist in the fact that a simple signal is detected without necessarily having recourse to markers, that the sensitivity of detection is improved, that there is less risk of obtaining false-negatives or false-positives and that the interpretation of the signals does not require an excessively complicated algorithm. Other advantages will appear below in the detailed description of the invention.

Thus, the present invention relates to a method for detecting a mutation at position n in a target nucleic acid, characterized in that it comprises the following steps.

a) hybridization of a probe linked in 5′ to a solid support of the DNA chip type with a target nucleic acid, the 3′ end of said probe hybridizing at most up to nucleotide n-1 of the target nucleic acid;

b) elongation of the probe hybridized in step a) by incorporation, in the 5′-3′ direction of nucleotides complementary to said target nucleic acid by means of a reaction mixture comprising at least one nucleotide derivative resistant to degradation by an exonuclease and a DNA polymerase,

c) digestion with said exonuclease such that only the probes elongated in step b) are not degraded, washing,

d) detection of the presence or absence of mutation by directly or indirectly measuring the presence or absence of DNA.

The key steps of this method are illustrated in the example presented in FIG. 1 below.

The expression “DNA polymerase” is understood to mean any natural or modified enzyme having a polymerase activity. There may be mentioned, for example, DNA pol exo-, in particular T7 or the Klenow fragment.

The expression “exonuclease” is understood to mean any natural or modified enzyme having an exonuclease activity. There may be mentioned, for example, exonuclease III. It is also possible to envisage the use of DNA polymerase possessing a pyrophophorolysis activity (in the presence of a high concentration of pyrophosphate, this enzyme adds a pyrophosphate to the last phosphodiester bond and therefore releases the nucleotide in 3′. This product is available from Promega under the trade mark READIT™, and variants using a system for visualizing luciferase is available under the trade mark READase™.

During step d), the presence or absence of mutation may be detected, according to a first embodiment, by measuring the modification of a property of the solid support linked to the presence or absence of DNA.

Another solution consists in detecting the presence or absence of mutation by optical reading of the presence or absence of DNA. The expression optical reading is understood to mean any measurement of absorption, transmission or emission of light which may optionally be at a specific wavelength (260 nm for example) either directly for the DNA, or for any marker molecule linked to the probe. This definition also comprises any measurement of fluorescence emitted by markers (fluorescein and/or phycoerythrin).

The expression “nucleotide derivative” is understood to mean any nucleotide analog which withstands degradation by a nuclease. There may be mentioned, for example, αS-phosphothioatedeoxynucleotides such as αS-dATP, αS-dTTP, αS-dCTP, αS-dGTP, αS-dUTP and αS-dITP. These nucleotide derivatives may be labeled, in particular with a fluorescent marker.

A “probe” is defined as being a nucleotide fragment comprising, for example from 10 to 100 nucleotides, in particular from 15 to 35 nucleotides, possessing a specificity of hybridization under defined conditions to form a hybridization complex with a target nucleic acid. The probes according to the invention, whether they are specific or nonspecific, may be immobilized, directly or indirectly, on a solid support and may carry a marker agent allowing or improving their detection.

Of course, the probe serves as a primer in the context of the invention since the objective is to incorporate a modified nucleotide at position n corresponding to the position of the mutation which is sought. The 3′ end of the probe therefore ends at the most and preferably at n-1.

The probe is immobilizable on a solid support by any appropriate means, for example by covalent bonding, by adsorption, or by direct synthesis on a solid support. These techniques are in particular described in patent application WO 92/10092.

The probe may be labeled by means of a marker chosen, for example, from radioactive isotopes, enzymes, in particular enzymes capable of acting on a chromogenic, fluorigenic or luminescent substrate (in particular a peroxidase or an alkaline phosphatase) or alternatively enzymes producing or using protons (oxidase or hydrolase); chromophoric chemical compounds, chromogenic, fluorigenic or luminescent compounds, nucleotide base analogs, and ligands such as biotin. The labeling of the probes according to the invention is carried out by elements selected from ligands such as biotin, avidin, streptavidin, dioxygenin, haptens, dyes, luminescent agents such as radioluminescent, chemiluminescent, bioluminescent, fluorescent and phosphorescent agents. Another possibility is to label the probe with a peptide comprising an epitope recognized by a given antibody. The presence of this antibody may be visualized by means of a second labeled antibody.

According to the first alternative mentioned above, step d) comprises the measurement of a variation of a physicochemical, electrical, optical or mechanical characteristic of the solid support in particular chosen from charge, doping, conductivity, resistance, impedance or any other effect of electrical variation, of the field effect or alternatively any variation of mass causing a variation of field, of the frequency of resonance or of electroacoustic admittance.

In this sense, the solid support consists of a DNA chip which may comprise a material selected from semi-conductors, dielectrics and piezoelectric transducers or a gold-prism structure. It is therefore possible to therefore find a basic structure of the Si/SiO² type, structures of the Metal-Oxide-Semiconductor (MOS), preferably Electrolyte-Oxide-Semiconductor (EOS), type. Such structures are described Jaffrezic-Renault N, ISFET-ENFET, Microcapteurs et Microtechniques [Microsensors and microtechniques] 225-235. In summary, this includes field effect transistors (FET), in particular ISFET, or preferably ENFET (Enzymatic Field Effect Transistor), type transistors. In the case of an ENFET support, it may be advantageous to link to the probe enzymes of the hydrolase or oxidase type which consume or produce protons. A substrate of these enzymes is added and the variation in pH is measured. Among the molecules which make it possible to improve and/or to simplify the detection, a group containing a metal atom may be grafted onto the probes, in particular a ferrocene group.

The expression measurement of a modification of the optical properties of the support is understood to mean any measurement of the variation of an optical property of the solid support linked to the presence or absence of DNA on said support. There may be mentioned, for example, the technology by the Biacore company which is in particular described in WO 97/38132. This embodiment of the invention therefore comprises the measurement of the refractive index of the support. It is possible to measure, by this technique, the internal and external reflection, for example ellipsometry, evanescent waves comprising the measurement of SPR (surface plasmon resonance), Brewster's angle of refraction, critical angle of reflection, FTR (frustrated total reflection), or STIR (scattered total internal reflection). These analyses may be carried out by means of Biacore 3000™.

In accordance with the second possibility mentioned above, step d) consists in measuring the quantity of light transmitted, absorbed or emitted. In this case, the support is made of a transparent material, in particular, glass. The techniques for the attachment of probes to glass are well known to persons skilled in the art. It is possible, for example, to measure the fluorescence of the probes labeled beforehand and to carry out the optical reading with a CCD camera.

In a preferred embodiment, an αS-phosphothioate-deoxynucleotide such as αS-dATP, αS-dTTP, αS-dCTP, αS-dGTP, αS-dUTP and αS-dITP is incorporated at the 3′ end of the probe.

This may be carried out for example by LCR, preferably by asymmetric PCR, the probe then serving as primer being in each case chemically coupled at its 5′ end to the solid phase at a predetermined site. The αS-phosphothioatedeoxynucleotides can be easily incorporated into polynucleotides by all the polymerases and reverse transcriptases tested, which makes it possible to use DNA polymerases having a more advantageous cost price than in other mutation detections.

Prior attachment of the probes at a determined site on the chip may be carried out by microfluidic targeting techniques developed by the company Orchid or photochemical targeting techniques by the company Attimetrix or alternatively electrotargeting by Cis-Bio international, said techniques being within the capability of persons skilled in the art.

According to the invention, the target DNA is hybridized with a probe so that its 3′ end immediately ends before the nucleotide to be identified. An αS-phosphothioatedeoxynucleotide is added to the 3′ end of the probe by means of a DNA polymerase and is consequently complementary to the nucleotide to be identified.

Step b) may be carried out in parallel on 4 sites (in tetrads) for each probe, with addition of a reaction mixture comprising a different αS-phosphothioate-dexoynucleotide per site. It is thus possible to detect a mutation in a specific position of the target DNA regardless of the nature of the base substitution. As regards the digestion of DNA in step c), exonuclease III may be advantageously used.

The method according to the invention is particularly intended for the detection of mutations in genes involved in diseases. There may be mentioned inherited genetic diseases, in particular hemochromatosis, sickle cell anemia, β and α thalassemias, cystic fibrosis, hemophilia, and mutations in the genes involved in cancer, for example in the Ras, p53 and BRCA1 genes. An exhaustive list of mutations in these genes is given at the following website: ftp://ncbi.nlm.nih.gov/repository/OMIM/morbidmap

In addition, the method according to the invention is useful during the study of the polymorphism of genes or of any genetic region and for the detection and/or identification of genetically modified organisms (GMO).

Another aspect of the invention relates to a device which makes it possible to carry out the method as described above. Such a method may comprise a system for detecting the presence or absence of DNA at a specific site of a chip, in particular a piezoelectric transducer, a field effect transducer, an optical density or fluorescence reader. It may be coupled to a data processing system, in particular to a computer.

Another aspect of the invention relates to a kit comprising a DNA chip to which there are attached probes and at least one of the elements chosen from:

a batch of 4 reaction mixtures each comprising a different αS-phosphothioatedexoynucleotide selected from αS-dATP, αS-dTTP, αS-dCTP and αS-dGTP,

a DNA polymerase,

an exonuclease, in particular exonuclease III,

a batch of solutions for solubilizing the DNA polymerase and/or the exonuclease in the case where these enzymes exist in the form of a freeze-dried powder.

Advantageously, the chips of this kit comprise a solid support of the ISFET or ENFET type.

This kit is intended for the detection of mutations of genes involved in diseases, in particular in inherited genetic diseases and in cancer. It can also serve for genetic typing and the study of the polymorphism of genes (for the detection of SNPs (Single Nucleotide Polymorphism)) and for the detection and/or identification of genetically modified organisms (GMO).

### LEGEND TO THE FIGURES

FIG. 1: schematic representation of a specific embodiment method according to the invention.

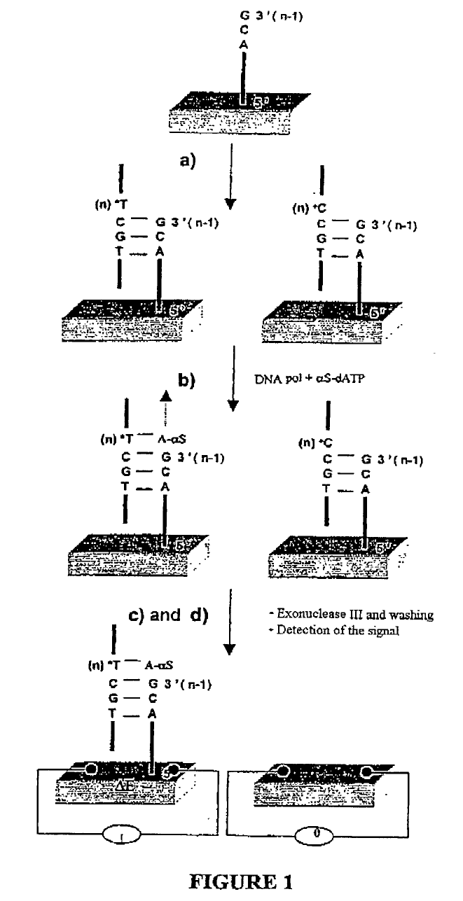

a) hybridization of a probe linked in 5′ to a solid support of the DNA chip type with a target nucleic acid, the 3′ end of said probe hybridizing up to nucleotide n-1 of the target nucleic acid,

b) incorporation in the 5′-3′ direction of an αS-dATP

c) digestion with exonuclease III so that only the probes elongated in step b) are not degraded, and washing,

d) detection of the presence or absence of the mutation by directly or indirectly measuring the presence or absence of DNA.

FIG. 2: conventional structure of a support of the Metal-Oxide-Semiconductor (MOS) type.

Diagrams taken from Jaffrezic-Renault.

FIG. 3: structures of the IFSET type.

A—IFSET taken from Jaffrezic-Renault

B—DNAFET

FIG. 4: principle of the chips according to the invention with a series of tetrads for the detection of mutations in the hemochromatosis gene.

### EXAMPLE 1

### Specific Embodiment of the Invention

The target DNA comprising a DNA fragment which contains a T→G mutation at position n to be identified, is added to the surface of the chip. Said DNA fragment hybridizes with the FITC (fluorescein isothiocyanate) labeled complementary oligonucleotide probe immobilized at a defined site on the support of the chip. During the subsequent reaction with polymerase, an incorporated phosphothioatedexoynucleotide (αSdATP) is present at a complementary position relative to the T nucleotide at position n. If the phosphothioatedexoynucleotide which is present in the reaction mixture is not complementary to the nucleotide to be identified (different from αSdATP), the probe is not extended in 3′. Exonuclease III then degrades all the probes which were not extended by a phosphothioatedexoynucleotide. The detection is then carried out by the binding of a conjugate anti-FITC conjugated with peroxidase. Once the enzyme-substrate reaction has taken place, a strong measurement signal therefore indicates if the nucleotide to be identified (T) is complementary to the phosphothioate-dexoynucleotide (αSdATP) which has been added to the reaction mixture for the reaction with polymerase.

### EXAMPLE 2

### Use of a Support of the ISFET or ENFET Type

(Jaffrezic-Renault N., Microcapteurs et Microtechniques 225-235)

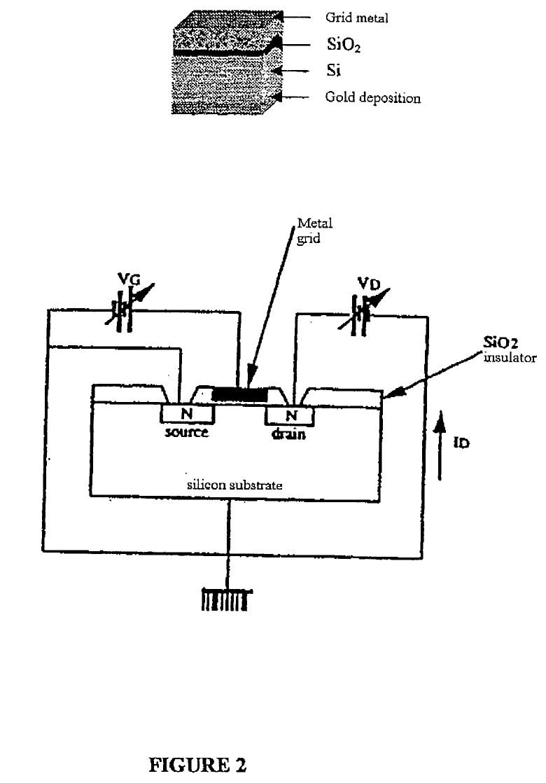

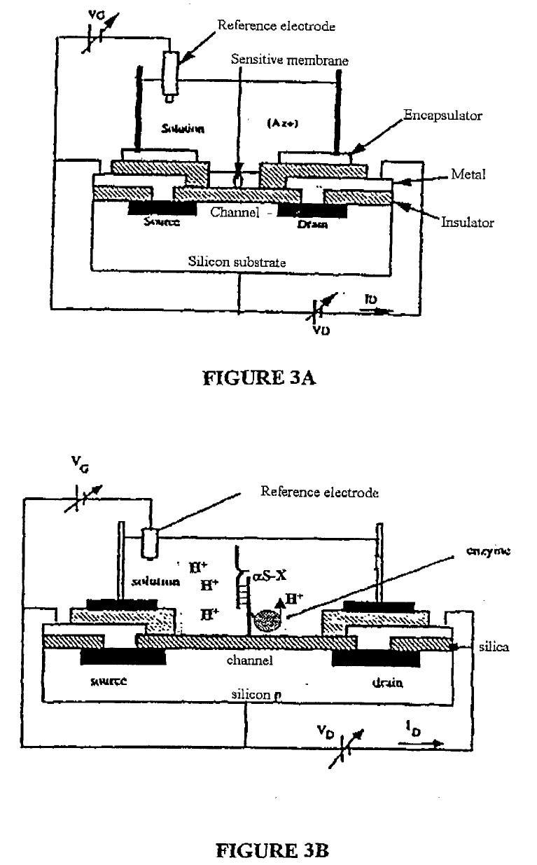

FIG. 3A (taken from Jaffrezic-Renault) schematically represents the ISFET structure. The latter is derived from the MOSFET structure (see FIG. 2; Jaffrezic-Renault) in this sense that the metal grid is replaced by the electrolytes and the reference electrode. dexoynucleotide The expression of the threshold voltage is:

V_{T}=Wsc−Wref+φ₀−(Qₛ+Q_{F})/Ci−2φ_{b}

V_{T} depends on the chemical characteristics of the solution (φ₀ is the potential difference between the sensitive membrane and the solution). In the circuit presented in FIG. 3A, the drain current is maintained constant and the variation in the voltage V_{G} which is proportional to φ₀ is measured.

The pH-sensitive membrane consists of thin layers of Al₂O₃, Ta₂O₅, Si₃N₄. Other membranes sensitive to the ions K⁺, Na⁻, Ag⁺, F⁻, Br⁻, I⁻, Ca₂⁺ and NO₃ are also available.

In the context of the invention, it is possible to attach to the support probes labeled with an enzyme which produces protons, ENFET system (FIG. 3B). A measurement is thus obtained of the presence or absence of DNA on the support following digestion with exonuclease III via a measurement of the variation of the pH of the solution, directly resulting in a variation in the voltage V_{T}. This system may be optionally coupled to one or more amplifier(s). The variation in voltage therefore denotes the presence of DNA. The system may be designed so that a voltage threshold variation causes or does not cause the passage of the current through a series of amplifiers and transistors and ultimately gives a binary type signal:

(1) variation in the voltage greater than or equal to the threshold of the transistor (DNA present and mutation detected),

(0) variation less than the threshold of the transistor (DNA absent and therefore no mutation).

These results can then be imported into a data processing system in order to compile the results obtained for each specific site on the chip.

### EXAMPLE 3

### Use of a Solid Support of the Piezoelectric Transducer Type

Certain materials such as SiO₂, TiO₃Ba, LiNbO₃ and the piezoelectric polymers (PVF2) have the property of bending when a physical stress is applied; Perrot H. and Hoummady M., Transducteurs pièzo-èlectrique [Piezoelectric transducers]. A measurable electric potential then appears due to the pressure exerted by the mass of DNA molecules. This measurement may be the resonance frequency or the admittance around the resonance frequency. In the case of the present invention, the DNA is present in a liquid medium, Consequently, it is also possible to measure the electroacoustic admittance or the conductivity which depends in particular on the density and the viscosity of the solution containing the electrolytes. It is thus possible to detect a difference of 100 pg in liquid medium.

### EXAMPLE 4

### Use of the Method According to the Invention for the Detection of Point Mutations Involved in Hemochromatosis

Point mutations designated HHP-1, HHP-19 and HHP-29 in U.S. Pat. No. 5,753,438 can be detected by means of the method according to the invention using a probe whose 3′ end terminates at n-1 from the position of the mutation:

HHP-1:

normal sequence

5′ TCTTTTCAGAGCCACTCACG₆₄CTTCCAGAGAAAGAGCCT 3′ (SEQ ID No. 1)

mutated sequence AG64

5′ TCTTTTCAGAGCCACTCACA₆₄CTTCCAGAGAAAGAGCCT 3′ (SEQ ID No. 2)

A probe having the sequence 5′ AGAAAAGTCTCGGTGAGTG₆₃ 3′ (SEQ ID No. 3) attached at 4 predefined sites of the chip (site A, T, G, C) is therefore used. At the site where the reaction mixture comprising αS-dTTP (site T) is applied, a signal is obtained in the case where there is indeed mutation in the DNA obtained from the sample. At the other sites A, G and C, no signal is obtained since the DNA is digested by exonuclease III.

For HHP-19 (A→G), the following probe may be used:

5′TATATAGATATTAGATATAAAGAA3′ (SEQ ID No. 4)

For HHP-29 (A→G), the following probe may be used:

5′AACCCCTAAAATATCTAAAAT3′ (SEQ ID No. 5)

It is also possible to detect the H63D mutation, which is due to the replacement of a cysteine with a guanine on the sense strand with the probes SED ID No. 6 and No. 7:

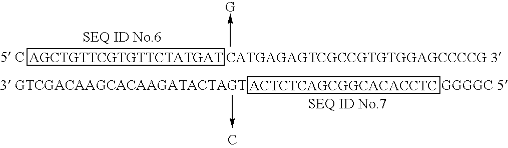

It is also possible to detect the mutation C282Y, which is due to the replacement of a guanine with an adenine on the sense strand, with the probes SED ID No. 8 and No. 9:

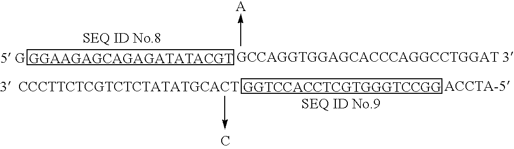

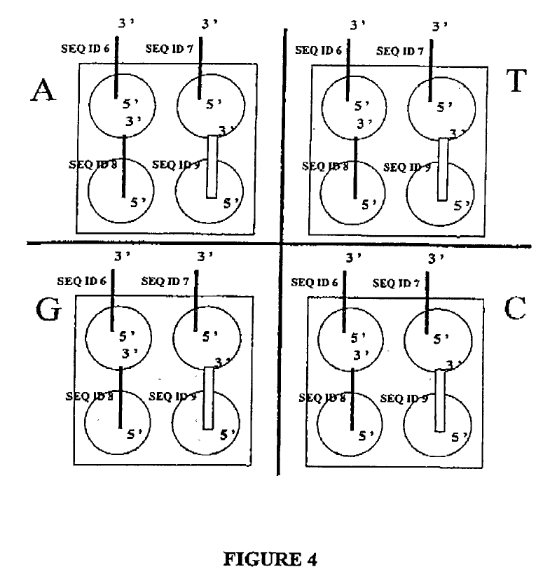

The four oligonucleotides SED ID No. 6 to 9 may be used for the identification of the nucleotide which is present immediately after the 3′ end of these oligonucleotides. A tetrad system may be provided to this effect (see FIG. 4).

### EXAMPLE 5

### Detection of Mutations in the Genes Involved in Cancer

a) Mutations in the MLH1 EST gene linked to the appearance of colorectal cancer.

There are currently 60 point mutations identified in MLH1 as being involved in colorectal cancer; Bronner (1994) Nature 368, 258, Papadopoulos (1994) Science 263, 1625.

The following mutations may be mentioned for example:

| Accession | Codon | Nucleotide | Amino acid |
|---|---|---|---|
| CM950799 | 62 | CAA-TAA | Gln-Term |
| CM960964 | 107 | ATA-AGA | Ile-Arg |

The complete list is given online at www.uwcm.ac.uk/uwcm/mg/ns/1/249617.html.

In the light of the large number of mutations for the same gene, the method of detection according to the invention with a chip comprising probes specific for each of the abovementioned mutations therefore appears essential to ensure a precise diagnosis for a patient.

b) Detection of mutations in the K-ras gene.

WO 91/13075 presents probes which make it possible to detect point mutations in codon 12 of K-ras. In the context of the invention, the following probes may be grafted onto the chip and consequently ensure complete detection of all possible mutations:

5′ AAGGCACTCTTGCCTACGCCA 3′ (SEQ ID No. 10)

5′ AGGCACTCTTGCCTACGCCAC 3′ (SEQ ID No. 11)

5′ AACTTGTGGTAGTTGGAGCT 3′ (SEQ ID No. 12)

5′ ACTTTGTGGTAGTTGGAGCTG 3′ (SEQ ID No. 13)

5′ ACTGGTGGTGGTTGGAGCAG 3′ (SEQ ID No. 14)

### EXAMPLE 6

1) Objective

The objective of this work is the determination of the genetic polymorphisin of a DNA using a novel biochip technique.

This technique consists in the genetic amplification of interest, the hybridization of the products of amplification obtained on a solid support (substrate) prepared beforehand by covalent attachment of a probe, extension of the probe with a modified nucleotide, visualization of degradation or protection of the probe.

This protocol described below is applied to the determination of the C282Y and H63D genotype of the hemochromatosis gene.

2) Protocol

a) Preparation of the DNA

PCR with primers to amplify genomic region of interest corresponding to the C282Y and H63D genotype.

Sequence of the primers used:

C282Y For: gggCTggATAACCTTggCT (SEQ ID No. 15)

C282Y Rev: gTCACATACCCCAgATCACA (SEQ ID No. 16)

H63D For: CCTTggTCTTTCCTTgTTTgA (SEQ ID No. 17)

H63D Rev: TCTggCTTgAAATTCTACTgg (SEQ ID No. 18)

These primers are modified at their 5′ end with the addition of a fluorescent marker Cy3 (Amersham)

The expected size of the amplification fragments in each case is about 100 bp

The amplifications obtained are checked on a 1.5% agarose gel

The probes used for C282Y and H63D are those described in example 4: two probes for each genotype to be determined.

b) Preparation of the solid supports

The probes are attached following a chemical modification of the surface allowing the reactivity of the 5′ ends of the probe oligonucleotides.

For each genotype to be determined, 2 probes are designed allowing the hybridization of the sense and anti-sense strands of the amplification, and positioned just upstream of the base to be visualized.

see description example 4.

c) Hybridization on the chips

1 Dilution of the amplifications volume:volume with 1X TE

2 Denaturation of the PCRs 100° C. for 5 min and then deposited on ice for 1 min

3 Hybridization of the PCRs:

Products of amplification diluted in a hybridization buffer (5X SSC, 1X Denhardt)

Deposition on the silicon substrate for the PCR

The substrates are placed in a humid room in a Petri dish at 37° C. for 45 min under 300 rpm

Washes with 5X SSC.

4 Detection of polymorphism:

a)—Elongation with dGTP alone

Mix produced for each chip:

Bst pol 8U/μl (Biolabs): 0.8 μl (that is 0.016 U/μl)

10X Bst pol buffer (Biolabs): 40 μl

alphaThiodGTP (Amersham) 1 mM:4 μl

Sterile water: 355.2 μl

Deposition of the 400 μl of mix on each chip

Incubation at 50° C. for 20 m in under 300 rpm

Washes with 5X SSC

b) Digestion

Mix produced for the 3 chips

Sterile water: 1 350 μl

10 X Exo III buffer (Biolabs): 150 μl

Exo III 100 U/μl (Biolabs): 0.3 μl

Deposition of the 400 μl of mix on each chip

Incubation at 37° C. for 10 min under 300 rpm

Washes in PBS 0.1% Tween 20

d) Visualization

The measurement of the fluorescence emission due to Cy3 is carried out (reading on a scanner for example) on each segment/chip.

3) Results

A positive signal was obtained for C282Y and H63D (results not presented).

For the various DNAs tested, a disappearance of the fluorescence due to Cy3 is observed in 1 segment out of 2 for the different genotypes.

In conclusion, degradation of some probes by Exo III is observed and this degradation is only observed for the strands which have not incorporated thiodGTP.

1. A method for detecting a mutation at position n in a target nucleic acid, characterized in that it comprises the following steps:
a) hybridization of a probe linked in 5′ to a solid support of the DNA chip type with a target nucleic acid, the 3′ end of said probe hybridizing at most up to nucleotide n-1 of the target nucleic acid;
b) elongation of the probe hybridized in step a) by incorporation, in the 5′-3′ direction of nucleotides complementary to said target nucleic acid by means of a reaction mixture comprising at least one nucleotide derivative resistant to degradation by an exonuclease and a DNA polymerase,
c) digestion with said exonuclease such that only the probes elongated in step b) are not degraded, washing,
d) detection of the presence or absence of mutation by directly or indirectly measuring the presence or absence of DNA.

2. The method as claimed in claim 1, characterized in that the presence or absence of mutation is detected in step d) by measuring the modification of a property of the solid support linked to the presence or absence of DNA.

3. The method as claimed in claim 1, characterized in that the presence or absence of mutation is detected in step d) by optical reading of the presence or absence of DNA.

4. The method as claimed in claim 2, characterized in that there is measured in step d) a variation of a physicochemical, electrical, optical or mechanical characteristic of the solid support in particular chosen from charge, doping, conductivity, resistance, impedance or any other effect of electrical variation, of the field effect or alternatively any variation of mass causing a variation of field, of the frequency of resonance or of electroacoustic admittance, of the refractive index of the support, in particular ellipsometry, of the evanescent waves comprising the measurement of SPR (surface plasmon resonance), of Brewster's angle of refraction, of the critical angle of reflection, FTR (frustrated total reflection), or STIR (scattered total internal reflection).

5. The method as claimed in claim 2, characterized in that the solid support consists of a DNA chip comprising a material selected from semi-conductors, dielectrics and piezoelectric transducers or a gold-prism structure.

6. The method as claimed in claim 5, characterized in that the solid support comprises structures of the Metal-Oxide-Semiconductor (MOS), preferably Electrolyte-Oxide-Semiconductor (EOS), type.

7. The method as claimed in claim 5, characterized in that the solid support comprises field effect transistors (FET), in particular ISFET or ENFET type transistors.

8. The method as claimed in claim 5, characterized in that a group containing a metal atom is grafted onto the probes, in particular a ferrocene group.

9. The method as claimed in claim 3, characterized in that step d) consists in measuring the quantity of light transmitted through the solid support, said support being made of a transparent material, in particular glass.

10. The method as claimed in claim 3, characterized in that step d) consists in measuring the fluorescence of the probes labeled beforehand.

11. The method as claimed in either of claims 9 and 10, characterized in that the optical reading is carried out by a CCD camera.

12. The method as claimed in one of claims 1 to 11, characterized in that there is used in step b) an αS-phosphothioatedexoynucleotide, preferably αS-dATP, αS-dTTP, αS-dCTP, αS-dGTP, αS-dUTP or αS-dITP.

13. The method as claimed in one of claims 1 to 12, characterized in that exonuclease III is used in step c).

14. The method as claimed in claims 1 to 13, characterized in that step b) is carried out in parallel on 4 sites for each probe, with addition of a reaction mixture comprising a different αS-phosphothioatedexoynucleotide per site.

15. The method as claimed in one of the preceding claims, intended for the detection of mutations of genes involved in diseases, in particular in inherited genetic diseases, in particular hemochromatosis, sickle cell anemia, β and α thalassemias, cystic fibrosis, hemophilia, and mutations in the genes involved in cancer.

16. The method as claimed in one of the preceding claims, intended for studying the polymorphism of genes or of any genetic region.

17. The method as claimed in one of the preceding claims, intended for the detection and/or identification of genetically modified organisms (GMO).

18. A device which makes it possible to carry out the method as claimed in one of the preceding claims.

19. A device as claimed in claim 18, characterized in that it comprises a system for detecting the presence or absence of DNA at a specific site of a chip, in particular a piezoelectric transducer, a field effect transducer, an optical density or fluorescence reader.

20. A kit comprising a DNA chip to which there are attached probes and at least one of the elements chosen from:
a batch of 4 reaction mixtures each comprising a different αS-phosphothioatedexoynucleotide selected from αS-dATP, αS-dTTP, αS-dCTP and αS-dGTP, αS-dUTP and αS-dITP,
a DNA polymerase,
an exonuclease, in particular exonuclease III,
a batch of solutions for solubilizing the DNA polymerase and/or the exonuclease in the case where these enzymes exist in the form of a powder.

21. The kit as claimed in claim 20, characterized in that the chips comprise a solid support of the ISFET or ENFET type.

22. The kit as claimed in either of claims 20 and 21, characterized in that it is intended for the detection of mutations of genes involved in diseases, in particular in inherited genetic diseases and in cancer.

23. The kit as claimed in either of claims 20 and 21, characterized in that it is intended for the detection of SNPs (Single Nucleotide Polymorphism).

24. The kit as claimed in either of claims 20 and 21, characterized in that it is intended for the detection and/or identification of genetically modified organisms (GMO).
